(19) United States
(12) Patent Application Publication
(10) Pub. No.: US 2023/0140921 A1
(43) Pub. Date: May 11, 2023

(54) METHOD FOR PROCESSING MEDICAL IMAGES

(71) Applicant: MISOINFO Tech., Seoul (KR)

(72) Inventors: Dong Uk An, Seoul (KR); Su-young Ho, Seoul (KR); Sang-do Nam, Seoul (KR); Jin-ho Son, Hanam-si (KR); Sang-don Byun, Daejeon (KR); Hyun-mok Song, Daejeon (KR); Gyeong-ho Kim, Daejeon (KR)

(21) Appl. No.: 17/983,293

(22) Filed: Nov. 8, 2022

(30) Foreign Application Priority Data
Nov. 9, 2021 (KR) 10-2021-0152744

(51) Int. Cl. G16H 30/40; G06F 21/57; G06F 21/62

(52) U.S. Cl. CPC G16H 30/40; G06F 21/577; G06F 21/6254; G06F 2221/034

(57) ABSTRACT

A method for processing a medical image performed by a computing device according to one aspect of the present disclosure. The method includes receiving a medical image of a patient and identifying one or more sensitive information from the received medical image; and creating the medical image as a safety image by de-identifying at least one of the sensitive information, wherein the sensitive information includes metadata of the medical image, personal information of the patient, and personal information of medical staffs related to the medical image.

### CROSS REFERENCE TO RELATED APPLICATION

The present application claims priority to Korean Patent Application No. 10-2021-0152744, filed on Nov. 9, 2021, the entire contents of which is incorporated herein for all purposes by this reference.

### BACKGROUND

### 1. Field

One aspect of the present disclosure relates to a method for processing an image, and more specifically, to a method of de-identifying a medical image.

### 2. Description of Related Art

In the era of the 4^{th}industrial revolution, leakage of personal information that may occur due to data exchange and transaction is being treated as a major problem. Accordingly, the image de-identification technology related to personal information protection is attracting attention, where the image de-identification refers to distortion or transformation in at least a part of an image so that the image cannot be identified. At home and abroad, research on de-identification by extracting features related to individuals in images using deep learning for data in various fields including the medical field is being actively conducted.

In particular, in the medical field, there are data closely related to personal information of patients, so the importance of de-identification technology for protecting personal information is further emphasized. Therefore, there is a need for a specific method for identifying sensitive information related to an individual within a medical image and de-identifying the identified sensitive information.

### RELATED ART DOCUMENTS

### Patent Documents

(Patent Document 0001) Korean Patent Registration No. 10-2167736

### SUMMARY

The present disclosure is devised in response to the above background art, and relates to a method for processing a medical image to prevent leakage of sensitive information.

The technical objects of the present disclosure are not limited to the technical objects mentioned above, and other technical objects not mentioned will be clearly understood by those skilled in the art from the following description.

In order to achieve the above objects, one aspect of the present disclosure discloses a method for processing medical images performed in a computing device according to one aspect of the present disclosure. The method may include receiving a medical image of a patient and identifying one or more sensitive information from the received medical image; and creating the medical image as a safety image by de-identifying at least one of the sensitive information, wherein the sensitive information may include metadata of the medical image, personal information of the patient, and personal information of medical staffs related to the medical image.

Alternatively, the medical image may include one or more sensitive information displayed as at least one of a text or an image.

Alternatively, the personal information of the patient may include a name of the patient, and the personal information of the medical staffs may include a name of the medical staffs in charge of the patient.

Alternatively, the metadata may include at least one of creation time information of the medical image, photographing location information, photographing equipment information, or information of an administrator who has created the medical image, the creation time information may indicate a time at which the medical image has been created, and the photographing location information may include at least one of an address of a location where the medical image is created or a name of the location.

Alternatively, identifying one or more sensitive information from the medical image may include: identifying texts included in the medical image using optical character recognition (OCR); and identifying one or more sensitive information and location information of the sensitive information based on the identified texts.

Alternatively, creating the medical image as a safety image may include: calculating a sensitivity representing a degree of risk to leak the sensitive information as a value when disclosed for each of the sensitive information; determining the sensitive information having the sensitivity greater than or equal to a predetermined threshold as risk information; and creating the safety image, in which the risk information is not identified in the medical image, by de-identifying the risk information.

Alternatively, de-identifying the risk information may include at least one of changing at least a part of the sensitive information or processing the sensitive information using a de-identifying filter.

Alternatively, the method may further include: creating a learning data set for training a neural network model based on the medical image; and training the neural network model using the learning data set, wherein the neural network model may include a model that identifies one or more sensitive information for the medical image and outputs the safety image.

Alternatively, creating the medical image as a safety image may include acquiring the safety image by inputting the medical image into the neural network model.

Alternatively, creating the medical image as a safety image may include: acquiring one or more location information of the sensitive information by inputting the medical image into the neural network model; and creating the safety image based on the location information of the sensitive information.

A computer program stored in a computer readable storage medium is disclosed according to one aspect of the present disclosure. The computer program, when executed on one or more processors, causes following operations for processing a medical image, and the operations may include: an operation of receiving a medical image of a patient, and identifying one or more sensitive information from the received medical image; and an operation of creating the medical image as a safety image by de-identifying at least one of the sensitive information, and wherein the sensitive information may include metadata of the medical image, personal information of the patient, and personal information of medical staff related to the medical image.

A computing device for processing a medical image is disclosed according to another aspect of the present disclosure, and the computing device may include: a memory; a network unit; and a processor, wherein the processor may be configured to: receive a medical image of a patient and identify one or more sensitive information from the received medical image; and create the medical image as a safety image by de-identifying at least one of the sensitive information, and the sensitive information may include metadata of the medical image, personal information of the patient, and personal information of medical staffs related to the medical image.

The technical solutions obtainable in the present disclosure are not limited to the above-mentioned technical solutions, and other technical solutions not mentioned will be clearly understood by those skilled in the art to which the present disclosure belongs from the description below.

The present disclosure can provide a medical image with a low risk of leakage of sensitive information.

The effects obtainable in the present disclosure are not limited to the above-mentioned effects, and other effects not mentioned will be clearly understood by those skilled in the art to which the present disclosure belongs from the description below.

### BRIEF DESCRIPTION OF DRAWINGS

Various aspects are now described with reference to the drawings, wherein like reference numerals are used to refer to like elements collectively. In the following aspects, for the purpose of explanation, numerous specific details are set forth in order to provide a thorough understanding of one or more aspects. However, it will be appreciated that such aspect(s) may be practiced without the specific details. In other examples, well-known structures and devices are shown in the form of block diagrams in order to facilitate the description of one or more aspects.

FIG. 1 is a schematic diagram illustrating a system in which various aspects of a computing device according to some aspects of the present disclosure may be implemented.

FIG. 2 is a block diagram of a computing device for processing a medical image according to some aspects of the present disclosure.

FIG. 3 is a schematic diagram illustrating a network function according to some aspects of the present disclosure.

FIG. 4A shows a first medical image that is an example of a medical image of the present disclosure.

FIG. 4B illustrates a first safety image obtained by de-identifying a first medical image according to some aspects of the present disclosure.

FIG. 4C illustrates a second safety image obtained by de-identifying a first medical image according to some aspects of the present disclosure.

FIG. 5A illustrates a second medical image that is another example of a medical image of the present disclosure.

FIG. 5B illustrates a third safety image obtained by de-identifying a second medical image according to some aspects of the present disclosure.

FIG. 6A illustrates a third medical image that is another example of a medical image of the present disclosure.

FIG. 6B illustrates a fourth safety image obtained by de-identifying a third medical image according to some aspects of the present disclosure.

FIG. 7 is a simplified general schematic diagram of an exemplary computing environment in which aspects of the present disclosure may be implemented.

### DETAILED DESCRIPTION

Various aspects are now disclosed with reference to the drawings. In the following description, numerous specific details are set forth to provide a thorough understanding of the present disclosure. However, it will also be appreciated that such aspects may be practiced without these specific details.

The terms “component,” “module,” “system,” and the like, as used herein, refer to a computer-related entity, hardware, firmware, software, a combination of software and hardware, or execution of software. For example, a component can be, but is not limited thereto, a procedure executed in a processor, a processor, an entity, a thread of execution, a program, and/or a computer. For example, both an application executed in a computing device and the computing device may be a component. One or more components may reside within a processor and/or thread of execution. One component may be localized within one computer. One component may be distributed between two or more computers. In addition, these components can be executed from various computer readable media having various data structures stored therein. For example, components may communicate via local and/or remote processes according to a signal having one or more data packets (for example, data from one component interacting with another component in a local system and a distributed system, and/or data transmitted via another system and a network such as an Internet through a signal).

In addition, the term “or” is intended to mean inclusive “or”, not exclusive “or”. In other words, unless otherwise specified or if unclear in context, the expression “X uses A or B” is intended to mean one of the natural inclusive substitutions. In other words, when X uses A, X uses B, or X uses both A and B, the expression “X uses A or B” can be applied to either of these cases. It is also to be understood that the term “and/or” used herein refers to and includes all possible combinations of one or more listed related items.

In addition, the terms “comprises” and/or “comprising” indicate the presence of corresponding features and/or elements, but do not exclude the presence or addition of one or more other features, components, and/or groups thereof. Further, unless otherwise specified or unless it is clear from the context to refer to a singular form, the singular in the specification and claims may generally be construed to refer to “one or more”.

Further, the term “at least one of A or B” has to be interpreted to refer to “including only A”, “including only B”, and “a combination of configurations of A and B”.

Those skilled in the art will further appreciate that the various illustrative logical blocks, configurations, modules, circuits, devices, logics, and algorithm steps described in connection with the aspects disclosed herein may be implemented in electronic hardware, computer software, or combinations of both. To clearly illustrate this interchangeability of hardware and software, various illustrative components, blocks, configurations, devices, logics, modules, circuits, and steps have been described above generally in terms of their functionality. Whether such functionality is implemented as hardware or software depends upon the specific application and design restrictions imposed on the overall system. Those skilled in the art may implement the described functionality in various ways for each specific application. However, such implementation decisions may not be interpreted as a departure from the scope of the present disclosure.

The description of the presented aspects is provided to enable any person skilled in the art to make or use the present disclosure. Various modifications to these aspects will be readily apparent to those skilled in the art. The generic principles defined herein may be applied to other aspects without departing from the scope of the present disclosure. Thus, the present disclosure is not intended to be limited to the aspects presented herein, but is to be construed in the widest scope consistent with the principles and novel features presented herein.

In the present disclosure, a network function, a neural network model, and a neural network may be used interchangeably.

The medical image of the present disclosure may be an image of a result of a medical examination for a patient, and may be an image including information on a medical field of a patient subject to medical analysis and diagnosis. For example, the medical image may be an X-ray image, an ultrasound examination result image, an electrocardiogram examination result image, or a body organ function examination result image of a patient. The body organ may be a lung, brain, liver, or the like, but the body organ of the present disclosure is not limited to the above-described examples. In addition, the above-described examples of the medical image are merely examples, and the medical image of the present disclosure is not be interpreted as being limited to the above-described examples.

The medical image of the present disclosure may include texts, where the texts may refer to texts included in a table or graph included in the medical image. Further, the medical image may be an image including one or more sensitive information displayed as at least one of a text and an image. That is, at least some of the texts included in the medical image may be sensitive information.

Sensitive information of the present disclosure may refer to information that infringes on personal privacy, and may be information that has a risk of leakage of personal privacy when sensitive information is disclosed. Specifically, the sensitive information of the present disclosure may be displayed as a text or an image, and may include metadata of a medical image, personal information of a patient, and personal information of a medical staff related to the medical image.

The personal information refers to information about an individual, capable of identifying the individual, for example, the personal information may be information about a name, age, or sex, but the personal information of the present disclosure is not limited to the above-described examples. The personal information of the patient of the present disclosure may include the name of the patient, and the personal information of the medical staff may include the names of one or more medical personnel in charge of the patient. In addition, the personal information of the patient may include not only the name of the patient, but also the age or sex of the patient, and the personal information of the medical staff may include not only the name of the medical staff, but also the ID or title, sex, and the like of the medical staff.

The metadata of the present disclosure may refer to metadata about a medical image, and may include at least one of information on creation time of the medical image, information on a photographing location, information on photographing equipment, and information of an administrator who has created the medical image.

The information on creation time of the medical image may refer to information indicating a creation time of the medical image, the information on a photographing location may refer to information indicating a location where the medical image is photographed, and the information on photographing equipment may refer to information indicating equipment that photographs a medical image or creates an image. In addition, the information on the administrator who has created the medical image may refer to information indicating the administrator who has created the medical image, and the administrator may refer to medical staffs who use the photographing equipment.

Specifically, the information on the creation time of the medical image may include at least one of a time at which the medical image is captured by the photographing equipment or a time at which the medical image is captured by the photographing equipment and transmitted to the administrator terminal. Further, the information on the photographing equipment may include information about a medical device that captures a medical image or a medical device that creates a medical image. The information on the administrator who has created the medical image may include information on the medical staff who uses the photographing equipment to capture the medical image. Further, the information on the photographing location may include at least one of an address of a photographing location where an image is created and a name of the photographing location. The photographing location may be a location in which the photographing equipment is located, and the information on the photographing location may include location information of the photographing equipment for photographing the medical image.

For example, the medical image may be an X-ray photograph obtained by photographing a chest of a patient. The metadata of the X-ray photograph may include creation time information indicating the date and time when the X-ray photograph has been created. In addition, the metadata of the X-ray photograph may include photographing equipment information including a model name or an identifier of X-ray equipment used for X-ray photographing. In addition, the metadata of the X-ray photograph may include photographing location information including at least one of a name of a hospital in which the X-ray has been photographed, or an address of the hospital. In addition, the metadata of the X-ray photograph may include information about the administrator including the user ID or name of the medical staff who has used the X-ray. The user ID may be an ID used when logging into software of the X-ray equipment.

The above-described medical image and metadata are only examples, and information included in the medical image or metadata of the present disclosure is not be interpreted as being limited to the above-described examples.

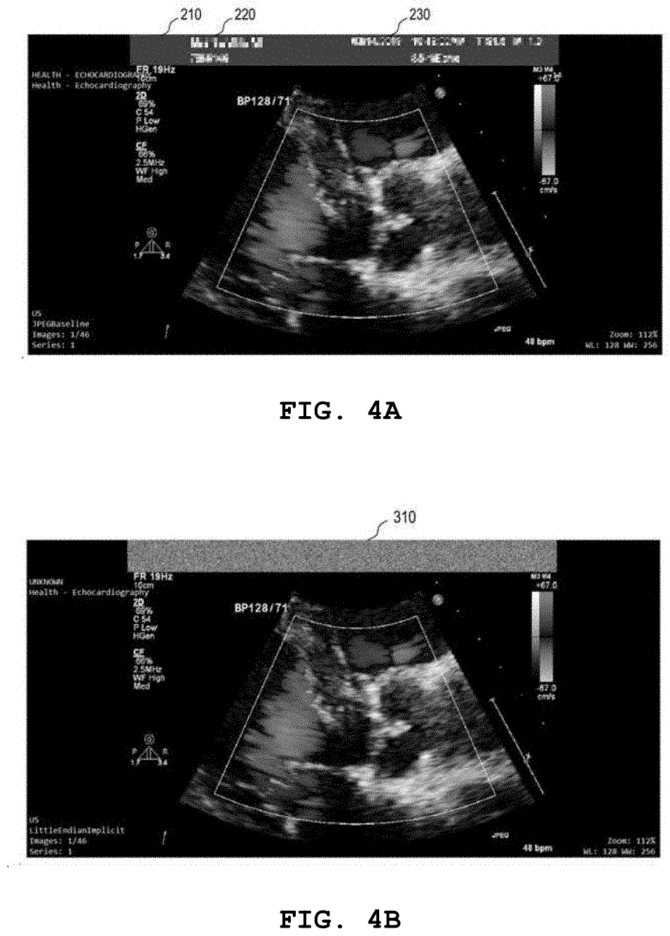

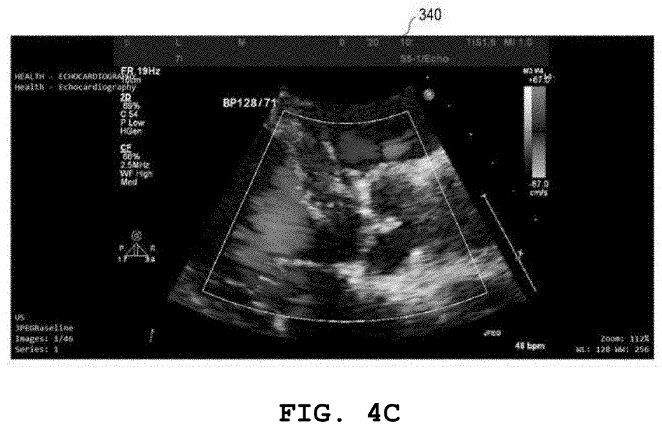

Examples of medical images of the present disclosure as described above are shown in FIGS. 4A, 5A, and 6A.

FIG. 4A shows a first medical image that is an example of a medical image of the present disclosure.

Specifically, FIG. 4A illustrates a first medical image showing the results of echocardiography of a patient. As shown in FIG. 4A, the first medical image may include texts and an echocardiogram, and some of the texts may be texts representing sensitive information.

The sensitive information included in the first medical image of FIG. 4A is photographing equipment information 210, creation time information 230 of the first medical image, and personal information of a patient 220. As shown in FIG. 4A, the photographing equipment information 210, the creation time information 230 of the first medical image, and the personal information 220 of a patient are displayed as texts. Specifically, the photographing equipment information 210 and the creation time information 230 may be included in the metadata of the first medical image. Referring to FIG. 4A, the photographing equipment information 210 indicates the name of a manufacturing company of photographing equipment, and the creation time information 230 indicates the date of Mar. 14, 2019 at which the first medical image has been created. In addition, the personal information 220 of a patient indicates the name of the patient.

The first medical image and sensitive information shown in FIG. 4A described above are only examples, and the medical image and sensitive information of the present disclosure are not limited to those shown in FIG. 4A.

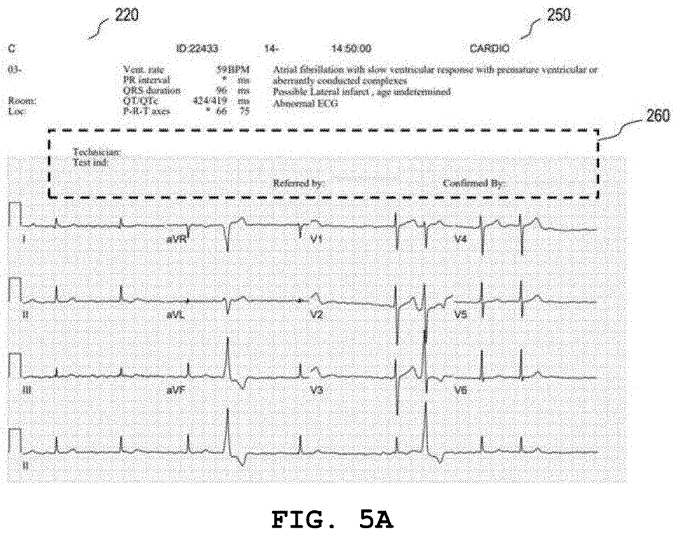

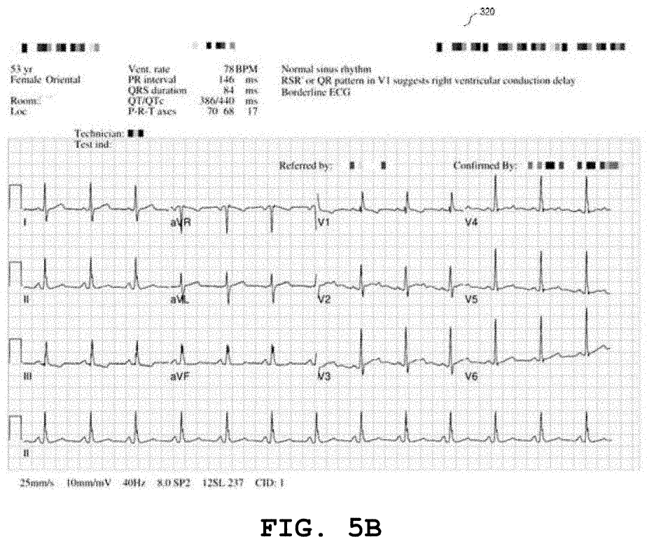

FIG. 5A illustrates a second medical image that is another example of the medical image of the present disclosure.

Specifically, FIG. 5A is a second medical image showing an electrocardiogram (ECG) result. As shown in FIG. 5A, the second medical image may include texts and ECG graphs.

In the second medical image of FIG. 5A, the sensitive information displayed as a text is personal information 220 of a patient, photographing location information 250, and personal information 260 of medical staffs. The personal information 220 of a patient indicates the name of the patient. In addition, the photographing location information 250 indicates the location where the second medical image has been photographed, and the personal information 260 of the medical staffs indicates the names of medical staffs who have used the photographing equipment that creates the second medical image and a doctor in charge of the patient.

The above-described second medical image and sensitive information shown in FIG. 5A are only examples, and the medical image and sensitive information of the present disclosure are not limited to those shown in FIG. 5a.

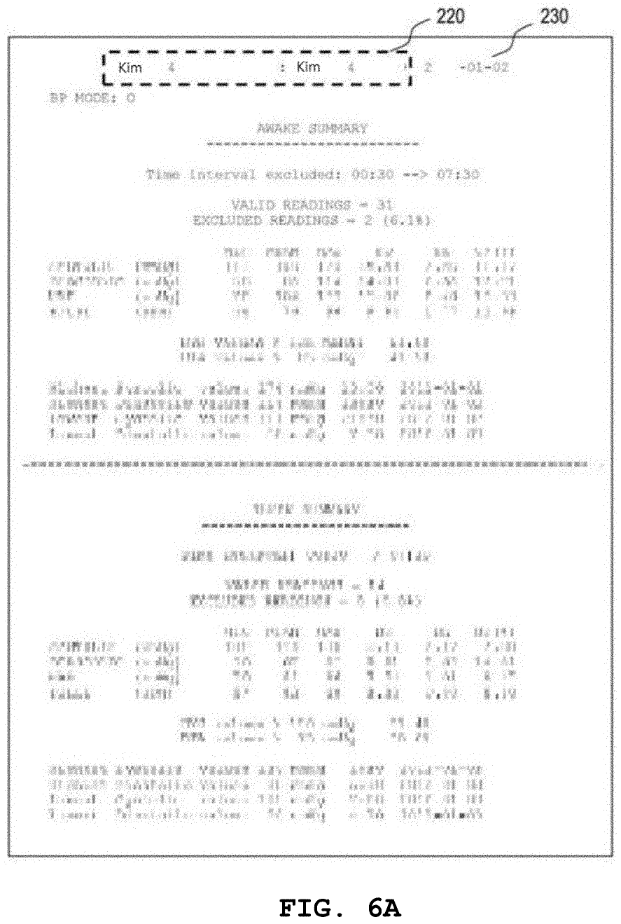

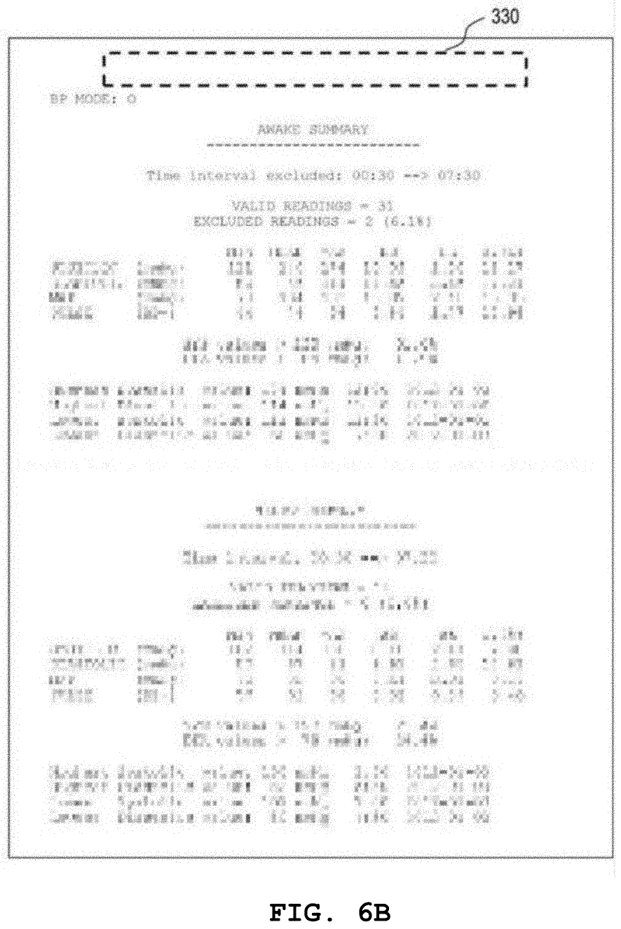

FIG. 6A illustrates a third medical image that is another example of the medical image of the present disclosure.

Specifically, FIG. 6A is a third medical image showing a function test result of a body organ. As shown in FIG. 6A, in the third medical image, various values measured in the functional test may be displayed as a text.

Sensitive information displayed as a text in the third medical image of FIG. 6aincludes creation time information 230 of the third medical image and personal information 220 of a patient. The personal information 220 of a patient indicates a preset identification ID and a name of the patient, and the creation time information 230 indicates the date of January 2, 2012 at which the third medical image has been created.

The third medical image and sensitive information shown in FIG. 6a described above are only examples, and the medical image and sensitive information of the present disclosure are not limited to those shown in FIG. 6A.

The data type of the medical image of the present disclosure may be DICOM, PNG, PDF, TXT, JPG, JPEG, GIF, or VCE, but the data type of the medical image of the present disclosure is not limited to the above-described examples.

In addition, the sensitive information of the present disclosure may be identified from the medical image using the optical character recognition (OCR) technique for the medical image or the character extraction technique within the image. In addition, in the present disclosure, one or more sensitive information may be identified in a medical image using a neural network model to be described below.

The location information of the sensitive information of the present disclosure may be information indicating a location where the sensitive information is displayed on a medical image. The location information of the sensitive information may be a coordinate value of a two-dimensional coordinate system expressed by setting the horizontal axis passing through the center of the medical image as the x-axis and the vertical axis as the y-axis.

For example, the sensitive information may be expressed as a coordinates such as (10,2). However, the sensitive information is not limited to the above-described coordinates or is not limited to be expressed as coordinates.

The safety image of the present disclosure may be an image with a lower risk in leakage of personal information compared to the original medical image. Specifically, the safety image of the present disclosure may be an image created by de-identifying one or more sensitive information included in the medical image, and the de-identified sensitive information may be information that does not visually display information properly. That is, the safety image of the present disclosure may be a medical image including de-identified sensitive information.

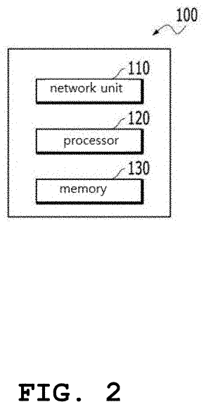

In addition, the sensitive information de-identified in the safety image of the present disclosure may be risk information of the present disclosure. Specifically, the risk information of the present disclosure may be information with a high risk of leakage of sensitive information when the corresponding sensitive information is disclosed, and may be sensitive information de-identified in a safety image. The degree of the risk to leak personal information may be indicated by the sensitivity in the present disclosure. That is, the risk information of the present disclosure may be determined based on the sensitivity of the sensitive information. A method of determining the risk information of the present disclosure and creating the safety image will be described in detail below with reference to FIG. 2.

The sensitivity in the present disclosure may be a value indicating a degree of a risk to leak the sensitive information when the corresponding sensitive information is disclosed. The sensitivity in the present disclosure may be variously expressed through a real number or a percentage. The sensitivity in the present disclosure may be a value calculated by a neural network model described below.

The de-identification processing in the present disclosure means the processing of information such that the content indicated by the information cannot be identified, and may be at least one of changing at least a part of the sensitive information or processing with a non-identification filter. Changing at least a part of the sensitive information means changing the displayed sensitive information to be different from the actual sensitive information.

As an example for changing at least a part of the sensitive information, the name of the patient included in the sensitive information may be ‘Hong Gil-dong’. When at least a part of the sensitive information is changed, the name of the patient of the sensitive information may be changed and displayed as ‘Hong Gil-soon’. As another example, changing at least a part of the sensitive information may be displaying the sensitive information by changing at least a part of the name of the patient to a special character such as an asterisk (*).That is, ‘Hong Gil-dong’, which is the name of the patient of the sensitive information, may be changed and displayed as ‘Hong**’.As another example, changing at least a part of the sensitive information may be displaying at least a part of the name of the patient by changing a color thereof. That is, only ‘Hong’ may be displayed while excluding ‘Gildong’ from the name of the patient ‘Hong Gil-dong’, which is the sensitive information, or the entire patient name may be displayed with a background color different from the text in the medical image. As another example, image processing may be applied to the name of the patient, Hong Gil-dong, so that the patient name may be blurred to the extent that it is difficult to identify.

The patient name of the sensitive information and de-identification processing types described above are only examples, and the sensitive information or de-identification processing types of the present disclosure are not limited to the above-described examples.

The color change of the present disclosure described in the above- examples is one of the de-identification processing types, and the de-identification processing may be performed by changing the color of at least a part of the sensitive information in the medical image. Specifically, as an example for color change, the sensitive information may be displayed by changing a color of at least a part of the sensitive information into a background color of the sensitive information. Displaying the sensitive information by excluding at least a part of the sensitive information will be described in detail below with reference to FIG. 6B.

The de-identification filter of the present disclosure may be an editing filter that may be applied to an image to de-identify at least a part of the image. According to aspects of the present disclosure, one or more de-identification filters may be applied to one medical image.

For example, the de-identification filter may be a filter related to one of blur processing, mosaic processing, or noise addition. The above-described non-identification filter is only examples, and the non-identification filter of the present disclosure is not limited to the above-described examples.

In addition, in the present disclosure, the safety level of the safety image may be different based on the application order or the number of applications of the non-identification filter. Specifically, the safety level of the present disclosure may be a value representing the degree of non-identification. According to some aspects of the present disclosure, the degree of safety of the created safety image may be calculated and represented as a value to indicate the safety level of the safety image against information leakage. The method of calculating the safety level of the present disclosure will be described in detail below with reference to FIG. 2.

Examples of the safety image of the present disclosure are shown in FIGS. 4B, 5B and 6B, and the safety image of the present disclosure will be described with reference to FIGS. 4B, 5B and 6B as follows.

FIG. 4B illustrates a first safety image obtained by de-identifying a first medical image according to some aspects of the present disclosure.

As described above, the sensitive information included in the first medical image of FIG. 4A includes the photographing equipment information 210, the creation time information 230 of the first medical image, and the personal information 220 of the patient. As shown in FIG. 4B, the first safety image may be a first medical image obtained by processing the photographing equipment information 210, the creation time information 230 of the first medical image, and the personal information 220 of the patient shown in FIG. 4A using the de-identification filter. Specifically, FIG. 4B shows an image to which noise 310 is added to the photographing equipment information 210, the creation time information 230 of the medical image, and the personal information 220 of the patient in the first medical image shown in FIG. 4A. In this case, the sensitive information of the first safety image of FIG. 4B is de-identified, so that the risk of leakage of sensitive information may be reduced compared to the first medical image of FIG. 4A.

The first safety image and noise addition 310 of FIG. 4B described above are only examples, and the de-identification filter or the safety image of the present disclosure may not be limited to those shown in FIG. 4B.

FIG. 5B illustrates a third safety image obtained by de-identifying the second medical image according to some aspects of the present disclosure.

As described above, the sensitive information displayed as a text in the second medical image of FIG. 5A includes the personal information 220 of the patient, the photographing location information 250, and the personal information 260 of medical staffs.FIG. 5B shows a third safety image obtained by mosaic-processing 320 of the personal information 220 of the patient, the photographing location information 250, and the personal information 260 of medical staffs in the second medical image of FIG. 5A. In this case, the sensitive information of the third safety image of FIG. 5B is de-identified, so that the risk of leakage of sensitive information may be reduced compared to the second medical image of FIG. 5A.

The third safety image and mosaic processing 320 of FIG. 5B described above are only examples, and the de-identification filter or the safety image of the present disclosure may not be limited to those shown in FIG. 5B.

FIG. 6B illustrates a fourth safety image obtained by de-identifying the third medical image according to some aspects of the present disclosure.

As described above, the sensitive information displayed as a text in the third medical image of FIG. 6A includes the creation time information 230 of the third medical image and the personal information 220 of the patient.FIG. 6A shows a fourth safety image obtained by color-changing 330 of the creation time information 230 and the personal information 220 of the patient of FIG. 6A.

Specifically, the creation time information 230 and the personal information 220 of the patient included in the third medical image are displayed as texts, and the colors of the image excluding the texts may have background colors. Accordingly, in FIG. 6A, color-changing 330 of the sensitive information is possible by changing the color of the texts of the creation time information 230 and the personal information 220 of the patient into the white color, which is the background color. In this way, the fourth safety image obtained by color-changing 330 of the sensitive information may be illustrated as shown in FIG. 6B.

In addition to the above, the de-identification processing of the present disclosure may include a crop processing or deletion processing that extracts or deletes only sensitive information from within the medical image, but the de-identification processing of the present disclosure is not limited to the above-described examples.

The fourth safety image and color-changing of FIG. 6B described above are only examples, and the de-identification filter or the safety image of the present disclosure may not be limited to those shown in FIG. 6B.

The data type of the safety image of the present disclosure may be DICOM, PNG, PDF, TXT, JPG, JPEG, GIF, or VCE, but preferably, the data type of the safety image of the present disclosure may be PNG. The data types described above are only examples, and the data types of the safety image of the present disclosure are not limited to the examples described above. Further, the data type of the safety image may be the same as or different from the data type of the medical image.

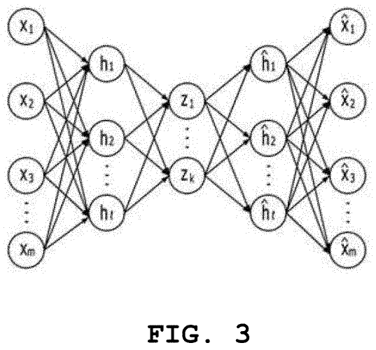

The neural network model of the present disclosure may be an artificial intelligence model that receives a medical image and outputs one or more each of sensitive information and location information of the sensitive information. The structure and training method of the neural network model of the present disclosure will be described in detail below with reference to FIG. 3.

The learning data set of the present disclosure is a set of data for training a neural network model. Specifically, the learning data set may be a set of data for training to output a safety image from the neural network model, calculate a sensitivity, or calculate location information for sensitive information when a medical image is input to the neural network model.

For example, the learning data set of the present disclosure may include a plurality of medical images in which at least one of sensitivity and sensitive information is labeled for supervised learning of the neural network model. As another example, the learning data set of the present disclosure may include a plurality of medical images for unsupervised learning or reinforcement learning of the neural network model.

The above-described learning data set is only examples, and the learning data set of the present disclosure is not limited to the above-described examples.

Hereinafter, a medical image processing method and a computing device for processing the medical image processing method according to some aspects of the present disclosure will be described.

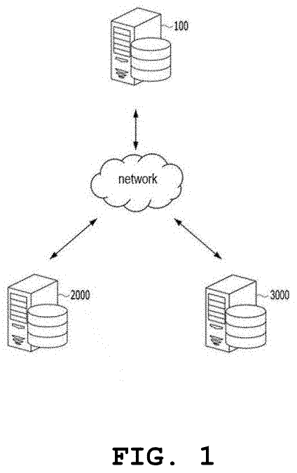

FIG. 1 illustrates a schematic diagram illustrating a system in which various aspects of a computing device according to some aspects of the present disclosure may be implemented.

A system according to aspects of the present disclosure may include a computing device 100, an external server 2000, medical equipment 3000, and a network. The computing device 100, the external server 2000, and the medical equipment 3000 according to aspects of the present disclosure may mutually transmit/receive data and signals for the system according to some aspects of the present disclosure through a network.

According to one aspect of the present disclosure, the medical equipment 3000 may refer to any type of entity(s) in a system having a mechanism for communication with the computing device 100. For example, the medical equipment 3000 may include any server implemented by at least one of an agent, an application programming interface (API), and a plug-in. In addition, the medical equipment 3000 may include an application source and/or a client application.

The medical equipment 3000 of the present disclosure may be photographing equipment that creates a medical image. Further, it may be a device capable of transmitting the created medical image to at least one of the external server 2000 or the computing device 100. Further, the medical equipment 3000 of the present disclosure may create the aforementioned metadata in relation to the created medical image. Specifically, the medical equipment 3000 of the present disclosure may create at least one of information about a location of the created medical image, information about a creation time of the medical image, information about the photographing equipment, or information of an administrator who has created the medical image.

The computing device 100 of the present disclosure may be integrated with the medical equipment 3000. That is, the components of the computing device 100 of the present disclosure, which will be described below with reference to FIG. 2, may be integrated into the medical equipment 3000, so that the medical image processing method may be performed in the medical equipment 3000. The medical equipment 3000 integrated with the computing device 100 may be photographing equipment capable of creating at least one of a medical image and a safety image.

The external server 2000 of the present disclosure may be a server or database of an external institution that collects information in the medical field. Specifically, the external server 2000 of the present disclosure may receive at least one of a medical image and a safety image from at least one of the medical equipment 3000 and the computing device 100, or vice versa.

For example, the external server 2000 may be a server or database of an external institution such as a bank, hospital, insurance company, public institution, etc., but the external institution related to the external server 2000 of the present disclosure is not limited to the above-described examples.

A detailed configuration of the computing device 100 of the present disclosure and technical features of each configuration will be described below in detail with reference to FIG. 2.

FIG. 2 is a block diagram of a computing device for processing a medical image according to one aspect of the present disclosure.

The configuration of the computing device 100 shown in FIG. 2 is only a simplified example. In one aspect of the present disclosure, the computing device 100 may include other components for performing the computing environment of the computing device 100, and only some of the disclosed components may configure the computing device 100.

The computing device 100 may include a network unit 110, a processor 120, and a memory 130.

The processor 120 may consist of one or more cores, and may include a processor for data analysis and deep learning of a central processing unit (CPU) of a computing device, a general purpose graphics processing unit (GPGPU), a tensor processing unit (TPU), and the like. The processor 120 may read a computer program stored in the memory 130 and perform data processing for machine learning according to one aspect of the present disclosure. According to one aspect of the present disclosure, the processor 120 may perform an operation for learning the neural network. The processor 120 may perform the calculation for learning the neural network, such as processing input data for learning in deep learning (DL), extracting features from input data, calculating an error, and updating the weight of the neural network using back propagation. At least one of a CPU, a GPGPU, and a TPU of the processor 120 may process the learning of a network function. For example, the CPU and the GPGPU together can process the learning of a network function and data classification using the network function. Further, in one aspect of the present disclosure, learning of a network function and data classification using the network function may be processed by using the processors of a plurality of computing devices together. In addition, the computer program executed in the computing device according to one aspect of the present disclosure may be a CPU, GPGPU or TPU executable program.

According to one aspect of the present disclosure, the memory 130 may store any type of information created or determined by the processor 120 and any type of information received by the network unit 110.

According to one aspect of the present disclosure, the memory 130 may include at least one type of storage media including a flash type memory, a hard disk type memory, a multimedia card micro type memory, a card type memory (for example, SD or XD memory, etc.), a random access memory (RAM). a static random access memory (SRAM), a read-only memory (ROM), an electrically erasable programmable read-only memory (EEPROM), a programmable read-only memory (PROM), a magnetic memory, a magnetic disk, and an optical disk. The computing device 100 may operate in relation to a web storage that performs a storage function of the memory 130 on the Internet. The description of the above memory is only an example, and the present disclosure is not limited thereto.

In the present disclosure, the network unit 110 may be configured regardless of a communication mode, such as a wired communication mode and a wireless communication mode, and may include various communication networks such as a local area network (LAN), a personal area network (PAN) and a wide area network (WAN). In addition, the network may be a well-known world wide web (WWW), and may use a wireless transmission technique used for short-range communication, such as infrared data association (IrDA) or Bluetooth.

The techniques described herein may be used in other networks as well as in the networks mentioned above.

According to one aspect of the present disclosure, the processor 120 of the present disclosure may receive a medical image of a patient for processing the medical image, and identify one or more sensitive information from the received medical image.

Specifically, the processor 120 may identify texts included in the medical image by using an optical character recognition (OCR) technology. Further, the processor 120 may identify one or more sensitive information and location information of the sensitive information based on the identified texts.

Specifically, the processor 120 may identify the texts included in the medical image by using a general OCR program executed as an OCR open source or the like. The processor 120 may identify one or more above-described sensitive information based on the identified texts. Identifying the sensitive information based on the texts may refer to extracting the sensitive information from the texts.

Referring to FIG. 4A, for example, the processor 120 may identify texts included in the medical image of FIG. 4A using an OCR open source. Accordingly, the processor 120 may extract one or more sensitive information, such as the photographing equipment information 210, the creation time information 230 of the medical image, and the personal information 220 of the patient based on the identified texts. Specifically, as shown in FIG. 4A, the medical image includes texts, and some of the texts may be texts indicating sensitive information. The processor 120 identifies texts indicating sensitive information among texts identified from the medical image, and based on at least some of the identified texts, the processor 120 can extract the sensitive information, such as the photographing equipment information 210, the creation time information 230, and the personal information 220 of the patient.

FIG. 4A and the sensitive information described above are only examples, and the texts, medical images or sensitive information of the present disclosure may not be limited to those shown in FIG. 4A.

In addition, the processor 120 may select at least a part of the identified sensitive information as risk information based on the predetermined reference risk information. The risk information may be the sensitive information that is de-identified in the safety image as described above, and the reference risk information may be at least a part of the sensitive information determined in advance to be de-identified in the safety image.

For example, the predetermined reference risk information is photographing equipment information, and the processor 120 may identify photographing equipment information and photographing location information from texts identified in the medical image. In this case, the processor 120 may select only the photographing equipment information as the safety information based on the predetermined reference risk information.

The above-described reference risk information and sensitive information are merely examples, and the reference risk information or sensitive information of the present disclosure is not limited to the above-described examples.

Further, the processor 120 may identify location information in which sensitive information composed of texts is located with respect to the medical image. The processor 120 may determine location information of texts, which correspond to sensitive information among the texts identified as described above, as location information of sensitive information.

According to one aspect of the present disclosure, the processor 120 may create a medical image as a safety image by de-identifying at least one of the sensitive information. Specifically, the processor 120 may calculate a sensitivity indicating the degree of risk to leak the sensitive information as a value when each sensitive information is disclosed. Further, the processor 120 may determine sensitive information having a sensitivity greater than or equal to a predetermined threshold as risk information.

Referring to FIG. 4A, for example, the processor 120 may determine the sensitivity for each of the photographing equipment information 210, the creation time information 230 of the medical image, and the personal information 220 of the patient, which are sensitive information identified in FIG. 4A.

When the sensitivity is expressed as a real number as described above, the processor 120 may calculate the sensitivity of the photographing equipment information 210 as 3, the sensitivity of the creation time information 230 as 4, and the sensitivity of the personal information 220 of the patient as 10. When the predetermined threshold is 5, the processor 120 selects the personal information 220 of the patient as the risk information from among the photographing equipment information 210, the creation time information 230 of the medical image, and the personal information 220 of the patient, which are the sensitive information.

FIG. 4A, sensitive information, and sensitivity described above are only examples, and the medical image, sensitive information or sensitivity of the present disclosure is not limited to those shown in FIG. 4A.

In addition, the processor 120 may create a safety image in which the risk information is not identified in the medical image by de-identifying the risk information. Specifically, as described above, the processor 120 may determine at least a part of the identified sensitive information as risk information based on at least one of the sensitivity or the reference risk information. In addition, the processor 120 may create the medical image as a safety image by performing the above-described de-identification processing on the risk information so that the selected risk information is not identified from the medical image.

Referring to FIGS. 5A and 5B, for example, the processor 120 may determine the personal information 220 of the patient, the photographing location information 250, and the personal information 260 of medical staffs as the risk information based on the sensitivity of each of the personal information 220 of the patient, the photographing location information 250, and the personal information 260 of medical staffs extracted from a part of the texts shown in FIG. 5A. The processor 120 may perform the mosaic-processing 320 to de-identify the personal information 220 of the patient, the photographing location information 250, and the personal information 260 of medical staffs, which are determined as risk information, so that the medical image of FIG. 5A can be created as the safety image shown in FIG. 5B.

FIGS. 5A and 5B, de-identification processing, sensitive information, and risk information described above are only examples, and the medical image, safety image, sensitive information, risk information or de-identification processing of the present disclosure is not limited to those shown in FIGS. 5A and 5B.

According to another aspect of the present disclosure, the processor 120 may create a learning data set for training the neural network model based on the medical image. Specifically, as described above, the processor 120 may create a learning data set for training the neural network model based on a medical image or a safety image. As described above, the learning data set may be created according to a learning method such as supervised learning, unsupervised learning, or reinforcement learning.

For example, the learning data set for supervised learning may include one or more sets matched with safety images and medical images, or one or more medical images labeled with location information of sensitive information. As another example, the learning data set for unsupervised learning may include at least one of medical images and safety images. The above-described learning data set is only examples, and the learning data set of the present disclosure is not limited to the above-described examples.

Further, the processor 120 may train the neural network model using the created learning data set. Specifically, the neural network model of the present disclosure may be an artificial neural network model that identifies texts through the text extraction or feature extraction from an image. The feature extraction may refer to a general feature extraction technique, for example, edge extraction or corner extraction of an image. In addition, the neural network model of the present disclosure may be trained by a learning method such as supervised learning, unsupervised learning, or reinforcement learning, and the trained neural network model of the present disclosure may be a model that identifies one or more sensitive information for a medical image and outputs a safety image.

According to another aspect of the present disclosure, the processor 120 may obtain a safety image by inputting a medical image to the neural network model. The processor 120 may acquire the above-described safety image by inputting the medical image to the neural network model trained as described above.

According to another aspect of the present disclosure, the processor 120 may obtain one or more location information of sensitive information by inputting a medical image to the neural network model. The neural network model may be a model that identifies one or more sensitive information for a medical image and outputs location information of each of the identified sensitive information. That is, the processor 120 may use the neural network model to obtain location information of sensitive information included in the medical image.

The processor 120 may create the above-described safety image based on location information of sensitive information obtained from the neural network model. Specifically, as described above, the processor 120 may create a safety image by determining at least a part of the sensitive information as risk information, and de-identifying the risk information based on the location information of the sensitive information.

According to another aspect of the present disclosure, the processor 120 may acquire the sensitivity of the identified sensitive information by inputting at least one of a medical image and sensitive information to the neural network model. Specifically, the processor 120 may identify sensitive information from the medical image, and input at least one of the medical image and the sensitive information to the neural network model to obtain the sensitivity of the sensitive information. That is, the processor 120 may use the neural network model to acquire the sensitivity of sensitive information included in the medical image. The processor 120 may determine risk information based on the sensitivity output from the neural network model and create a safety image as described above.

The processor 120 of the present disclosure may calculate a safety level for a safety image created according to some aspects of the present disclosure. Specifically, the processor 120 may calculate a safety level indicating a leakage degree or an identification level of sensitive information as a value based on the safety image created as described above. The processor 120 of the present disclosure may determine to perform additional de-identification processing on the safety image based on the safety level of the safety image. Specifically, the safety level of the safety image of the present disclosure will be described with reference to FIGS. 4A, 4B and 4C as follows.

FIG. 4C illustrates a second safety image obtained by de-identifying the first medical image according to some aspects of the present disclosure.

FIG. 4C shows a second safety image in which the photographing equipment information 210, the creation time information 230 of the first medical image, and the personal information 220 of the patient, which are sensitive information compared to the first medical image of FIG. 4A, are not identified. The second safety image may be an image obtained by changing the text color of sensitive information using the aforementioned non-identification filter.

However, when the second safety image of FIG. 4C is compared with the first safety image of FIG. 4C, it can be seen that the first safety image of FIG. 4B is more difficult to identify sensitive information than the second safety image of FIG. 4C. Accordingly, the processor 120 may calculate the second safety level of the second safety image of FIG. 4C to have a value lower than a value of the first safety level of the first safety image of FIG. 4B. For example, the processor 120 may calculate the first safety level of the first safety image of FIG. 4B as 10 and the second safety level of the second safety image of FIG. 4C as 5.

FIGS. 4A to 4C, the first and second safety images, the first medical image, the de-identification processing, the safety level, and the sensitive information described above are only examples, and the safety image, the medical image, the de-identification processing, the safety level, and the sensitive information of the present disclosure are not limited to those shown in FIGS. 4A to 4C.

Medical images used in the field of medical research expose sensitive information irrelevant to the research. According to the present disclosure, even if the safety image is publicly used for research in the research field, the risk of leakage of sensitive information is remarkably low, so it has a remarkable effect in terms of safety. Therefore, the present disclosure may have a remarkable effect that it is possible to provide a safety image which is safe against leakage of sensitive information through de-identification processing of the medical image.

In addition, the present disclosure may also have another remarkable effect that it is possible to specifically indicate the safety of information leakage of the medical image by calculating the degree of exposure of sensitive information as a safety level with respect to the de-identification processing performed on the medical image.

Hereinafter, the configuration of the neural network model for processing a medical image and the learning method according to some aspects of the present disclosure will be described.

FIG. 3 is a schematic diagram illustrating a network function according to one aspect of the present disclosure.

Throughout the specification, a neural network model, a calculation model, a neural network, a network function, and a neural network may be used interchangeably. The neural network may be composed of a set of interconnected calculation units, which may generally be referred to as nodes. These nodes may also be referred to as neurons. The neural network is configured to include at least one or more nodes. Nodes (or neurons) constituting the neural network may be interconnected by one or more links.

In the neural network, one or more nodes connected through a link may relatively form a relationship between an input node and an output node. The concept of the input node and the output node is relative, and any node serving as an output node with respect to one node may serve as an input node with respect to another node, and vice versa. As described above, an input node-to-output node relationship may be created about a link. One or more output nodes may be connected to one input node through a link, and vice versa.

In the relationship between the input node and the output node connected to each other through one link, the value of the data of the output node may be determined based on data input to the input node. A link that interconnects the input node and the output node may have a weight. The weight may be variable, and may be changed by the user or algorithm in order to allow the neural network to perform a desired function. For example, when one or more input nodes are interconnected to one output node by respective links, the output node may determine the output node value based on the values input to the input nodes connected to the output node and the weight assigned to the links corresponding to the respective input nodes.

As described above, one or more nodes are interconnected through one or more links in the neural network, thereby forming the relationship between the input node and an output node in the neural network. The characteristics of the neural network may be determined according to the number of nodes and links in the neural network, the correlation between the nodes and the links, and the value of a weight assigned to each of the links. For example, when there are two neural networks including the same number of nodes and links and having different weight values of the links, the two neural networks may be recognized as they are different from each other.

The neural network may consist of a set of one or more nodes. A subset of nodes constituting the neural network may constitute a layer. Some of the nodes constituting the neural network may configure one layer based on distances from the initial input node. For example, a set of nodes having a distance n from the initial input node may constitute n layers. The distance from the initial input node may be defined by the minimum number of links required to pass there through to reach the corresponding node from the initial input node. However, the definition of such a layer is arbitrary for description, and the order of the layer in the neural network may be defined in a different way from the above. For example, a layer of nodes may be defined by a distance from the final output node.

The initial input node may refer to one or more nodes to which data is directly input without going through a link in a relationship with other nodes among nodes in the neural network. Alternatively, in a relationship between nodes based on a link in a neural network, it may mean nodes that do not have other input nodes connected by a link. Similarly, the final output node may refer to one or more nodes that do not have an output node in a relationship with other nodes among nodes in the neural network. In addition, a hidden node may mean nodes constituting the neural network other than the first input node and the final output node.

The neural network according to one aspect of the present disclosure may be a neural network in which the number of nodes in the input layer may be the same as the number of nodes in the output layer, and the number of nodes decreases and then increases again from the input layer to the hidden layer. In addition, the neural network according to another aspect of the present disclosure may be a neural network in which the number of nodes in the input layer may be less than the number of nodes in the output layer, and the number of nodes decreases from the input layer to the hidden layer. In addition, the neural network according to another aspect of the present disclosure may be a neural network in which the number of nodes in the input layer may be greater than the number of nodes in the output layer, and the number of nodes increases from the input layer to the hidden layer. The neural network according to another aspect of the present disclosure may be a neural network which is a combination of the aforementioned neural networks.

A deep neural network (DNN) may refer to a neural network including a plurality of hidden layers in addition to an input layer and an output layer. The deep neural network can be used to identify the latent structures of data. In other words, it can identify the potential structure of photos, texts, videos, voices, and music (e.g., what objects are in the photos, what the text and emotions are, what the texts and emotions are, etc.).The deep neural network may include convolutional neural networks (CNNs), recurrent neural networks (RNNs), auto encoders, generative adversarial networks (GANs), and restricted Boltzmann machines (RBMs), deep belief networks (DBNs), Q networks, U networks, Siamese networks, and generative adversarial networks (GANs).The above description of the deep neural network is only an example, and the present disclosure is not limited thereto.

In one aspect of the present disclosure, the network function may include an auto encoder. The autoencoder may be a kind of artificial neural networks for outputting output data similar to input data. The autoencoder may include at least one hidden layer, and an odd number of hidden layers may be disposed between the input/output layers. The number of nodes in each layer may be reduced from the number of nodes of the input layer to the number of nodes of an intermediate layer called a bottleneck layer (encoding), and then expanded from the bottleneck layer to the output layer (symmetrical with the input layer)symmetrically with the reduction. The autoencoder can perform non-linear dimensionality reduction. The number of input layers and output layers may correspond to a dimension after preprocessing the input data. In the autoencoder structure, the number of nodes of the hidden layer included in the encoder may decrease as it moves away from the input layer. If the number of nodes in the bottleneck layer (the layer with the fewest nodes located between the encoder and decoder) is too small, a sufficient amount of information may not be delivered, so the number of nodes is maintained at a certain number or more (e.g., half or more of the input layer, etc.).

The neural network may be trained using at least one of supervised learning, unsupervised learning, semi-supervised learning, and reinforcement learning. The training of the neural network may be a process of applying knowledge, which allows the neural network to perform a specific operation, to the neural network.

The neural network may be trained in a way that minimizes output errors. The training for the neural network refers to the process of iteratively inputting the learning data into the neural network, calculating the output of the neural network and the target error for the learning data, and updating the weight of each node of the neural network by back-propagating the error of the neural network from the output layer of the neural network to the input layer in the direction to reduce the error. In the case of supervised learning, learning data in which the correct answer is labeled in each learning data is used (that is, labeled learning data), and in the case of unsupervised learning, the correct answer may not be labeled in each learning data. That is, for example, learning data in the case of supervised learning regarding data classification may be data in which categories are labeled for each of the learning data. Labeled learning data is input to the neural network, and an error can be calculated by comparing the output (category) of the neural network with the label of the learning data. As another example, in the case of unsupervised learning regarding data classification, an error may be calculated by comparing the input learning data with the neural network output. The calculated error is back propagated in the reverse direction (that is, from the output layer to the input layer) in the neural network, and the connection weight of each node of each layer in the neural network may be updated according to the back propagation. A change amount of the connection weight of each node to be updated may be determined according to a learning rate. The calculation of the neural network on the input data and the back propagation of errors may constitute a learning cycle (epoch). The learning rate may be applied differently depending on the number of repetitions of the learning cycle of the neural network. For example, in the early stage of learning of a neural network, a high learning rate can be used to enable the neural network to quickly acquire a certain level of performance, thereby increasing efficiency, and a low learning rate can be used at the end of learning to increase the accuracy.

In the learning of the neural network, in general, the learning data may be a subset of real data (that is, data to be processed using the learned neural network), and thus there is a learning cycle in which the error on the learning data is reduced, but the error on the real data is increased. Overfitting refers to a phenomenon in which errors on actual data increase by over-learning on learning data as described above. An example of the overfitting is a phenomenon in which a neural network that has learned a cat by seeing a yellow cat does not recognize a cat when it sees a cat having a color other than yellow. The overfitting may act as a cause of increasing errors in machine learning algorithms. In order to prevent such overfitting, various optimization methods can be used. In order to prevent the overfitting, methods such as increasing the learning data, regularization, dropout for deactivating some of the nodes of the network in the process of learning, and the use of a batch normalization layer can be applied.

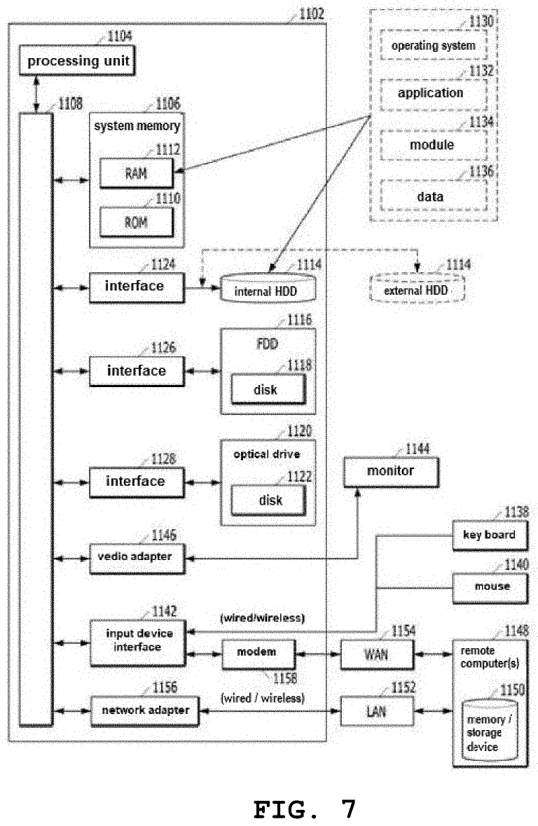

FIG. 7 illustrates a simple general schematic diagram of an example computing environment in which aspects of the present disclosure may be implemented.

Although the present disclosure has been described above in that it can be implemented by the computing device, those skilled in the art will appreciate that the present disclosure may be implemented with computer-executable instructions that may be executed on at least one computer and/or as a combination of hardware and software and/or in combination with other program modules.

In general, program modules include routines, programs, components, data structures, etc. that may perform specific tasks or implement specific abstract data types. In addition, those skilled in the art will appreciate that the methods of the present disclosure can be implemented not only with single-processor or multiprocessor computer systems, minicomputers, and mainframe computers, but also with other computer system configurations including personal computers, handheld computing devices, microprocessor-based or programmable consumer electronics, etc. (each of which can be operated in connection with one or more associated devices).

The aspects described in the present disclosure may also be practiced in distributed computing environments where certain tasks are performed by remote processing units that are linked through a communications network. In a distributed computing environment, program modules may be located in both local and remote memory storage devices.

Computers typically include a variety of computer-readable media. Media accessible by a computer may be computer readable media regardless the type thereof, and the media accessible by a computer may include volatile and nonvolatile media, transitory and non-transitory media, removable and non-removable media. By way of an example, but not limited thereto, computer-readable media may include computer-readable storage media and computer-readable transmission media.

Computer-readable storage media include volatile and non-volatile media, temporary and non-transitory media, removable and non-removable media implemented in any method or technology for storing information such as computer readable instructions, data structures, program modules or other data. Computer-readable storage media may include, but not limited thereto, RAMs, ROMs, EEPROMs, flash memory or other memory technologies, CD-ROMs, digital video disks (DVDs) or other optical disk storage devices, magnetic cassettes, magnetic tapes, magnetic disk storage devices or other magnetic storage devices, or any other media that can be accessed by a computer and used to store desired information.

Computer readable transmission media typically implement computer readable instructions, data structures, program modules or other data in a modulated data signal such as a carrier wave or other transport mechanism, and include any information delivery medium. The term ‘modulated data signal’ refers to a signal in which one or more characteristics of the signal are set or changed so as to encode information in the signal. By way of an example, but not limited thereto, computer-readable transmission media include wired media such as a wired network or direct-wired connection, and wireless media such as acoustic, RF, infrared, and other wireless media. Combinations of any of the above are also intended to be included within the scope of computer-readable transmission media.

An example environment 1100 including a computer 1102 for implementing various aspects of the disclosure is shown, and the computer 1102 includes a processing unit 1104, a system memory 1106, and a system bus 1108. The system bus 1108 connects system components including (but not limited thereto) the system memory 1106 to the processing unit 1104. The processing unit 1104 may be any of a variety of commercially available processors. A dual processor and other multiprocessor architectures may also be used as the processing unit 1104.

The system bus 1108 may be any of several types of bus structures that may further be interconnected to a memory bus, a peripheral device bus, and a local bus using any of a variety of commercial bus architectures. The system memory 1106 includes a read only memory (ROM) 1110 and a random access memory (RAM) 1112. A basic input/output system (BIOS) is stored in a non-volatile memory 1110, such as a ROM, an EPROM, an EEPROM, etc., and the BIOS may include a basic routine that helps transmission of information between components within the computer 1102, such as during startup. The RAM 1112 may also include high-speed RAM, such as static RAM for caching data.

The computer 1102 may also include an internal hard disk drive (HDD) 1114 (for example, EIDE, SATA) - this internal hard disk drive 1114 may also be configured for external use within a suitable chassis (not shown) -, a magnetic floppy disk drive (FDD) 1116 (for example, for reading from or writing to removable diskette 1118), and an optical disk drive 1120 (for example, for reading from or writing to a CD-ROM disk 1122 or for reading from or writing to other high capacity optical media such as a DVD). The hard disk drive 1114, the magnetic disk drive 1116, and the optical disk drive 1120 may be connected to the system bus 1108 by a hard disk drive interface 1124, a magnetic disk drive interface 1126, and an optical drive interface 1128, respectively. The interface 1124 for implementing the external drive may include at least one or both of Universal Serial Bus (USB) and IEEE 1394 interface technologies.

These drives and their associated computer-readable media provide non-volatile storage of data, data structures, computer-executable instructions, and the like. In the case of the computer 1102, drives and media correspond to one that stores any data in a suitable digital format. Although the computer readable media are described based on HDDs, removable magnetic disks, and removable optical media such as CDs or DVDs, those skilled in the art will be appreciated that other computer-readable media such as zip drives, magnetic cassettes, flash memory cards, cartridges, and the like may also be used in the exemplary operating environment, and any such media may include computer-executable instructions for performing the methods of the present disclosure.

A number of program modules including operating systems 1130, one or more application programs 1132, other program modules 1134, and program data 1136 may be stored in the drive and the RAM 1112. All or portions of the operating systems, applications, modules, and/or data may also be cached in the RAM 1112. It will be appreciated that the present disclosure may be implemented in various commercially available operating systems or combinations of operating systems.

A user may input commands and information into the computer 1102 via one or more wired/wireless input devices, for example, a pointing device such as a keyboard 1138 and a mouse 1140. Other input devices (not shown) may include a microphone, an IR remote controller, a joystick, a game pad, a stylus pen, a touch screen, and the like. Although these and other input devices are often connected to the processing unit 1104 through the input device interface 1142 that is connected to the system bus 1108, parallel ports, IEEE 1394 serial ports, game ports, USB ports, IR interfaces, and the like may be connected by other interfaces.

A monitor 1144 or other type of display devices is also coupled to the system bus 1108 via an interface such as a video adapter 1146. In addition to the monitor 1144, the computer generally includes other peripheral output devices (not shown) such as speakers, printers, and the like.

The computer 1102 may operate in a networked environment using logical connections to one or more remote computers such as remote computer(s) 1148 via wired and/or wireless communications. The remote computer(s) 1148 may refer to workstations, computing device computers, routers, personal computers, portable computers, microprocessor-based entertainment devices, peer devices, or other common network nodes, and may generally include many or all of the components described with respect to the computer 1102, but only the memory storage device 1150 is shown for simplicity. The logical connections shown in the drawings include wired/wireless connections to a local area network (LAN) 1152 and/or a larger network, for example, a wide area network (WAN) 1154. Such LAN and WAN networking environments are common in offices and companies, and facilitate enterprise-wide computer networks, such as intranets, and all of which can be connected to a worldwide computer network, for example, the Internet.

When used in the LAN networking environment, the computer 1102 is connected to the local network 1152 through a wired and/or wireless communication network interface or an adapter 1156. The adapter 1156 may facilitate the wired or wireless communication to the LAN 1152, which also includes a wireless access point installed therein for communicating with a wireless adapter 1156. When used in a WAN networking environment, the computer 1102 may include a modem 1158, may be connected to a communication computing device on the WAN 1154, or may include other devices for establishing communications over the WAN 1154. A modem 1158, which may be an internal or external and wired or wireless device, is coupled to the system bus 1108 via the serial port interface 1142. In a networked environment, program modules described with respect to the computer 1102 or portions thereof may be stored in a remote memory/storage device 1150. It will be appreciated that the network connections shown in the drawings are exemplary and other devices for establishing a communication link between the computers may be used.

The computer 1102 may communicate with any wireless devices or entities that are operated through wireless communication, such as printers, scanners, desktop and/or portable computers, portable data assistants (PDAs), communication satellites, and any devices or place, and phones in association with wireless detectable tags. It may include at least Wi-Fi and Bluetooth wireless technologies. Accordingly, the communication may be a predefined structure as in a conventional network or may simply be an ad hoc communication between at least two devices.

Wi-Fi (Wireless Fidelity) makes it possible to connect to the Internet, etc. without a wire. The Wi-Fi refers to a wireless technology such as cell phones that allow these devices, for example, computers, to transmit and receive data indoors and outdoors, that is, anywhere within the coverage area of a base station. The Wi-Fi networks use a radio technology called IEEE 802.11 (a, b, g, etc.) to provide safe, reliable, and high-speed wireless connections. The Wi-Fi can be used to connect computers to each other, to the Internet, and to wired networks (using IEEE 802.3 or Ethernet). The Wi-Fi networks may operate in unlicensed 2.4 and 5 GHz radio bands, for example, at 11 Mbps (802.11a) or 54 Mbps (802.11b) data rates, or in products that include both bands (dual band).

Those skilled in the art of the present disclosure will understand that information and signals may be represented using any of various different technologies and techniques. For example, data, instructions, commands, information, signals, bits, symbols, and chips that may be referenced from the above description may be expressed by voltages, currents, electromagnetic waves, magnetic fields or particles, optical field particles or particles, or any combination thereof.

A person having ordinary skill in the art of the present disclosure will recognize that various illustrative logical blocks, modules, processors, means, circuits and algorithm steps described in connection with the aspects disclosed herein may be implemented by electronic hardware (referred to as software for the purpose of convenience), various types of program, design codes, or a combination thereof. To clearly explain the interchangeability of hardware and software, various illustrative components, blocks, modules, circuits, and steps have been described above generally in terms of their functionality. Whether such functionality is implemented as hardware or software depends upon the specific application and design restrictions imposed on the overall system. A person skilled in the art of the present disclosure may implement the described functionality in various ways for each specific application, and such implementation decisions may not be interpreted as a departure from the scope of the present disclosure.

The various aspects presented herein may be implemented as methods, apparatuses, standard programming and/or articles of manufacture using engineering techniques. The term article of manufacture includes computer programs, carriers or media accessible from any computer-readable storage device. For example, the computer-readable storage medium includes magnetic storage devices (for example, hard disks, floppy disks, magnetic strips, etc.), optical disks (for example, CDs, DVDs, etc.), smart cards, and flash memory devices (for example, EEPROMs, cards, sticks, key drives, etc.), but it is not limited thereto. In addition, various storage media presented herein include one or more devices for storing information and/or other machine-readable media.

It is to be understood that the specific order or hierarchy of steps in the presented processes is an example of exemplary approaches. It is to be understood that the specific order or hierarchy of steps in the processes within the scope of the present disclosure may be rearranged based on design priorities.

The appended method claims present elements of the various steps in a sample order, but are not limited to the presented specific order or hierarchy.

The description of the presented aspects is provided to enable any person skilled in the art to make or use the present disclosure. Various modifications to these aspects will be readily apparent to those skilled in the art, and the generic principles defined herein may be applied to other aspects without departing from the scope of the present disclosure. Thus, the present disclosure is not intended to be limited to the aspects presented herein, but is to be construed in the widest scope consistent with the principles and novel features presented herein.

What is claimed is:

1. A method for processing a medical image performed by a computing device, the method comprising:
receiving a medical image of a patient and identifying one or more sensitive information from the received medical image; and
creating the medical image as a safety image by de-identifying at least one of the sensitive information,
wherein the sensitive information includes metadata of the medical image, personal information of the patient, and personal information of medical staffs related to the medical image.

2. The method of claim 1, wherein the medical image includes one or more sensitive information displayed as at least one of a text or an image.

3. The method of claim 1, wherein the personal information of the patient includes a name of the patient, and
the personal information of the medical staffs includes a name of the medical staffs in charge of the patient.

4. The method of claim 1, wherein the metadata include at least one of creation time information of the medical image, photographing location information, photographing equipment information, or information of an administrator who has created the medical image,
the creation time information indicates a time at which the medical image has been created, and
the photographing location information includes at least one of an address of a location where the medical image is created or a name of the location.

5. The method of claim 1, wherein identifying one or more sensitive information from the medical image includes:
identifying texts included in the medical image using optical character recognition (OCR); and
identifying one or more sensitive information and location information of the sensitive information based on the identified texts.

6. The method of claim 1, wherein creating the medical image as a safety image includes:
calculating a sensitivity representing a degree of risk to leak the sensitive information as a value when disclosed for each of the sensitive information;
determining the sensitive information having the sensitivity greater than or equal to a predetermined threshold as risk information; and
creating the safety image, in which the risk information is not identified in the medical image, by de-identifying the risk information.

7. The method of claim 1, wherein de-identifying the risk information includes at least one of changing at least a part of the sensitive information or processing the sensitive information using a de-identifying filter.

8. The method of claim 1, further comprising:
creating a learning data set for training a neural network model based on the medical image; and
training the neural network model using the learning data set,
wherein the neural network model includes a model that identifies one or more sensitive information for the medical image and outputs the safety image.

9. The method of claim 8, wherein creating the medical image as a safety image includes acquiring the safety image by inputting the medical image into the neural network model.

10. The method of claim 8, wherein creating the medical image as a safety image includes:
acquiring one or more location information of the sensitive information by inputting the medical image into the neural network model; and
creating the safety image based on the location information of the sensitive information.

11. A computer program stored in a computer readable storage medium, wherein the computer program, when executed on one or more processors, causes following operations for processing a medical image, and the operations include:
an operation of receiving a medical image of a patient, and identifying one or more sensitive information from the received medical image; and
an operation of creating the medical image as a safety image by de-identifying at least one of the sensitive information, and
wherein the sensitive information includes metadata of the medical image, personal information of the patient, and personal information of medical staff related to the medical image.

12. A computing device for processing a medical image, the computing device comprising:
a memory;
a network unit; and
a processor,
wherein the processor is configured to:
receive a medical image of a patient and identify one or more sensitive information from the received medical image; and
create the medical image as a safety image by de-identifying at least one of the sensitive information, and
the sensitive information includes metadata of the medical image, personal information of the patient, and personal information of medical staffs related to the medical image.
